(19) United States
(12) Patent Application Publication
(10) Pub. No.: US 2019/0076008 A1
(43) Pub. Date: Mar. 14, 2019

(54) ENDOSCOPE LIGHT SOURCE, CONTROL METHOD OF ENDOSCOPE LIGHT SOURCE, AND ENDOSCOPE APPARATUS

(71) Applicant: SONY CORPORATION, TOKYO (JP)

(72) Inventors: MITSUNORI UEDA, TOKYO (JP); TOMOYUKI OKI, KANAGAWA (JP); TAKASHI YAMAGUCHI, KANAGAWA (JP); AKIO FURUKAWA, TOKYO (JP); SATORU MIZOUCHI, KANAGAWA (JP); HIROTAKA MURAMATSU, KANAGAWA (JP)

(21) Appl. No.: 16/083,889

(22) PCT Filed: Jan. 24, 2017
(86) PCT No.: PCT/JP2017/002284

§ 371 Date: Sep. 10, 2018

(30) Foreign Application Priority Data
Mar. 18, 2016 (JP) 2016-054977

(51) Int. Cl. A61B 1/06; A61B 1/07; G02B 23/26; G02B 23/24

(52) U.S. Cl. CPC A61B 1/0661; A61B 1/07; A61B 1/063; A61B 1/0638; G02B 23/2476; G02B 23/2469; G02B 23/26

(57) ABSTRACT

[Object] To propose an endoscope light source capable of making an area of a region to be irradiated with illumination light changeable, a control method of an endoscope light source, and an endoscope apparatus using such an endoscope light source. [Solution] An endoscope light source according to the present disclosure includes: a light source section that emits light from at least one or more solid light sources; a coupling section capable of connecting with a light guide connected to an endoscope; and a control section that performs control so as to make an incident angle of a light ray that enters the light guide in the coupling section, changeable.

### TECHNICAL FIELD

This disclosure relates to an endoscope light source, a control method of an endoscope light source, and an endoscope apparatus.

### BACKGROUND ART

In recent years, various medical actions have been being performed using an endoscope apparatus disclosed in, for example, Patent Literature 1 shown below.

One of such the medical actions includes laparoscopic surgery and thoracoscopic surgery using rigid endoscopes in place of laparotomy and open chest surgery. Although these surgeries using rigid endoscopes are said to be less invasive for patients, for doctors being surgeons, there are many difficulties, such as strangulation of a visual field, lack of a stereoscopic effect, interference between other surgical instruments and camera due to working in narrow space, and interference with illumination. However, in recent years, along with the miniaturization and high definition of imaging elements, making an imaging area to a wider angle becomes to be also realized. Accordingly, it becomes possible to increase a distance to an imaging object. With this, while looking the same picture as that in the past, it has become possible to execute working in a space much wider than that in the past.

Moreover, as one of other medical actions using endoscope apparatuses, there is observation of luminal organs by using flexible endoscopes. In the case where an image acquired by a flexible endoscope with regard to luminal organs is displayed on a display screen, the organs located at the back side are displayed on the center portion of the screen, and the organs located at the front side with a close distance from the flexible endoscope are displayed on a peripheral portion of the screen.

### CITATION LIST

### Patent Literature

Patent Literature 1: JP 5750422B

### DISCLOSURE OF INVENTION

### Technical Problem

However, in the laparoscopic surgeries and thoracoscopic surgeries using rigid endoscopes, in the case where a distance to an imaging object is larger, an area to be illuminated becomes also wider. Accordingly, in order to perform working under the same brightness as that in the past, a light source brighter than that in the past becomes necessary (for example, if a distance to an imaging object becomes twice that in the past, the area to be illuminated becomes four times that in the past). Hitherto, as illumination of endoscope apparatuses, it is common to use a xenon (Xe) lamp. However, with such a Xe lamp, there is no margin in brightness, and it is not possible to cope with the above situations. Moreover, even if a light source with more high luminance is realized, a peripheral portion of a region to be noticed as a surgical region will also be irradiated with illumination light, which leads to that a waste occurs in illumination light.

Moreover, in observation for luminal organs using a flexible endoscope, since an image of a peripheral portion is near in distance to the illumination of a flexible endoscope, it is whitened brightly. However, for organs located on the back side, illumination may become insufficient. If the intensity of illumination on the back side is raised simply, the illuminance at organs in the vicinity becomes too high, and there is a possibility that the tissues of the organs in the vicinity may be heated by the illumination light.

Thus, at the time of medical actions using endoscope apparatuses, a technique is required that can improve the utilization efficiency of illumination light by making an area of a region to be irradiated with illumination light changeable.

Then, in this disclosure, in view of the above-mentioned circumstances, proposed are an endoscope light source capable of making an area of a region to be irradiated with illumination light changeable, a control method of an endoscope light source, and an endoscope apparatus using such an endoscope light source.

### Solution to Problem

According to the present disclosure, there is provided an endoscope light source, including: a light source section that emits light from at least one or more solid light sources; a coupling section capable of connecting with a light guide connected to an endoscope; and a control section that performs control so as to make an incident angle of a light ray that enters the light guide in the coupling section, changeable.

In addition, according to the present disclosure, there is provided a control method of an endoscope light source, including: guiding a light ray emitted from a light source section that emits light from at least one or more solid light sources, to a coupling section capable of connecting with a light guide connected to an endoscope, and changing an incident angle of a light ray that enters the light guide in the coupling section.

In addition, according to the present disclosure, there is provided an endoscope apparatus, including: an endoscope that is inserted in an inside of an examination object, images an inside of the examination object, and propagates an obtained captured image to a display apparatus; a light source section that emits light from at least one or more solid light sources as illumination light used when the endoscope images an inside of the examination object; a coupling section capable of connecting with a light guide connected to the endoscope; and a control section that performs control so as to make an incident angle of a light ray that enters the light guide in the coupling section, changeable.

According to the present disclosure, light rays emitted from a light source section are guided to a coupling section, and the incident angle of the light rays that enter a light guide is controlled in the coupling section.

### Advantageous Effects of Invention

As described in the above, according to the present disclosure, it becomes possible to make an area of a region to be irradiated with illumination light changeable in an endoscope light source, and it is possible to improve the utilization efficiency of illumination light.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram showing schematically an entire constitution of an endoscope apparatus according to an embodiment of the present disclosure.

FIG. 2 is an explanatory diagram showing schematically a detailed constitution of an endoscope light source according to the embodiment.

FIG. 3 is an explanatory illustration showing schematically one example of a light source section included in an endoscope light source according to the embodiment.

FIG. 4A is an explanatory illustration for describing Etendue.

FIG. 4B is an explanatory illustration for describing Etendue.

FIG. 5 is an explanatory illustration for describing Etendue.

FIG. 6 is an explanatory illustration for describing a control process of an incident angle of light rays to a light guide in an endoscope light source according to the embodiment.

FIG. 7A is an explanatory diagram showing schematically a constitution of a coupling section included in an endoscope light source according to the embodiment.

FIG. 7B is an explanatory diagram showing schematically a constitution of a coupling section included in an endoscope light source according to the embodiment.

FIG. 7C is an explanatory diagram showing schematically a constitution of a coupling section included in an endoscope light source according to the embodiment.

FIG. 8 is an explanatory illustration showing schematically the first concrete example of the coupling section according to the embodiment.

FIG. 9 is a graph chart showing a relationship between an incident angle of light rays to a light guide and a radiation angle direction from the light guide.

FIG. 10 is an explanatory illustration showing schematically the second concrete example of the coupling section according to the embodiment.

FIG. 11 is an explanatory illustration showing schematically the third concrete example of the coupling section according to the embodiment.

FIG. 12 is an explanatory illustration showing schematically the fourth concrete example of the coupling section according to the embodiment.

FIG. 13 is an explanatory illustration showing schematically the fifth concrete example of the coupling section according to the embodiment.

FIG. 14 is an explanatory illustration showing schematically the fifth concrete example of the coupling section according to the embodiment.

FIG. 15 is an explanatory illustration showing schematically the fifth concrete example of the coupling section according to the embodiment.

FIG. 16 is an explanatory illustration showing schematically the sixth concrete example of the coupling section according to the embodiment.

FIG. 17A is an explanatory illustration showing schematically the sixth concrete example of the coupling section according to the embodiment.

FIG. 17B is an explanatory illustration showing schematically the sixth concrete example of the coupling section according to the embodiment.

FIG. 18 is an explanatory illustration showing schematically the seventh concrete example of the coupling section according to the embodiment.

FIG. 19 is a flow diagram showing one example of a flow of a control method of an endoscope light source according to the embodiment.

FIG. 20 is a flow diagram showing another example of a flow of a control method of an endoscope light source according to the embodiment.

FIG. 21 is an explanatory diagram showing schematically a modified example of an endoscope apparatus according to the embodiment.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

It should be noted that description will be given in the following order.

1. Study with regard to endoscope light source

2. Embodiment
2.1. With regard to entire constitution of endoscope apparatus
2.2. With regard to constitution of endoscope light source
2.3. With regard to control method of endoscope light source
2.4. With regard to modified example of endoscope apparatus

3. Conclusion

(Study with Regard to Endoscope Light Source)

In advance of description with regard to an endoscope light source, an endoscope apparatus, and a control method of the endoscope light source, the contents of the study by the present inventors with regard to the endoscope light source are shown, and an object of the present disclosure is described in detail.

As mentioned before, in recent years, various medical actions have been being performed using an endoscope apparatus. As such medical actions, in place of laparotomy and open chest surgery, there are laparoscopic surgery and thoracoscopic surgery using rigid endoscopes, and observation of luminal organs by using flexible endoscopes.

Although it is said that these surgeries using rigid endoscopes are less invasive for patients, for doctors acting as surgeons, there are many difficulties, such as strangulation of a visual field, lack of a stereoscopic effect, interference between other surgical instruments and camera due to working in narrow space, and interference with illumination.

With regard to strangulation of a visual field, although optical systems have been devised so as to be able to obtain an observation range with a field angle being as wide as possible, in the case of using imaging elements with the same pixel size, since an observation region per one pixel becomes large, lowering of resolution is caused. However, in recent years, the miniaturization and high definition of the imaging elements have been accomplished, and a so-called high definition (High Definition: HD) image (so-called 2K image quality) has been put in practical use widely. Moreover, a 4K imaging system to create a 4K image and a display apparatus corresponding to the 4K imaging system also have been put in practical use. Furthermore, experiments using a super Hi-Vision (8K) endoscope with resolution higher than that of a 4K image has been executed.

Along with realization of the above-described high definition imaging systems, in the endoscope apparatuses, the wider angle of a photographing region has also come to be realized. With the higher definition of the imaging system, the following two advantages has come to be acquired.

In the endoscope apparatus with the narrow view field in the past, in the case of enlarging a view, the endoscope has been brought close to an object. However, as the first advantage, along with the higher definition of the imaging system, in a condition that the position of an endoscope main body is kept as it is, it becomes possible to use the endoscope as an enlarging microscope that enlarges electrically the center portion of a captured image. Understandably, in the case of enlarging electrically more than a certain limit, since pixels become rough, the practical use as an enlarged view is not acquired. However, in the imaging system with high definition of 4K or more, in the case where the center of a screen is electronically enlarged by about two times to 10 times, sufficient usefulness can be acquired. With this, it becomes possible to perform fine surgery using an endoscope. Moreover, a display screen (monitor) of an endoscope apparatus becomes to have a function as a microscope.

As the second advantage, along with the higher definition of an imaging system, for example, in order to acquire an image of the definition obtained in the past by photographing with the 2K imaging system, in the 4K imaging system, pixels of one quarter of it become sufficient. That is, in the case of an imaging system with the same field angle, it is possible to double a distance to an object. Accordingly, while watching the same picture as that in the past, it becomes possible to secure a much wider space than that in the past. However, in the case where a wide space has been realized, the matter that a distance from illumination becomes two times, means that an area to be illuminated becomes four times. That is, in order to illuminate in the inside of a space with the same brightness as that in the past, it is required to use a light source that is four times brighter than the present situation.

In the past, as the illumination used for an endoscope, it is common to use the illumination using a Xe lamp of 500 W. However, in the brightness of it, there is no margin to an extent to illuminate all of a wide space with sufficient brightness. Therefore, in order to realize brightness being four times higher than that in the past, it is required to realize a light source with still high luminance. Moreover, even if such a very bright light source has been realized, in the case of displaying an enlarged image on a display screen, an image on a peripheral portion of a region to be noticed becomes unnecessary. For example, in the case of viewing by enlarging a 2K range of a central portion of a 4K image, the area of a peripheral portion occupies 75% of the whole area. Accordingly, with regard to the brightness of illumination, heat, electric power for light emission in a light source, and the like, the most of them is used for the illumination for the peripheral portion. That is, unless a light source is made to have higher brightness than that in the present situation, a wide surgical space cannot be secured. However, even if the wide surgical space has been realized, the most of light becomes useless at the time of an actual surgical operation.

In view of such the present situation, if illumination is realized in such a way that an illumination region of a light source changes in accordance with a zooming operation (i.e., viewing by enlarging an image), the present inventors have considered that it becomes possible to use separately two types of functions of (1) in the case of securing a wide surgical space while keeping the luminance as it is in the present state, and (2) in the case of obtaining an enlarged image by realizing a wide view field in a distance, similar near that in the past, between a rigid endoscope and an object.

Moreover, generally, in many cases, an object to be observed using a flexible endoscope of medical application is luminal organs. In the case of displaying an acquired image on a display screen etc., luminal organs located on the back side are displayed on the center portion of a screen, and on the peripheral portion of the screen, wall surfaces of luminal organs located at the closest distance from the endoscope are displayed. Since the peripheral portion of the screen is near from the illumination of an endoscope, it is whitened brightly. However, for the luminal organs located on the back side, the illuminance may become insufficient. If an operation to raise the illuminance on the back side is executed simply, the illuminance on the wall surfaces of luminal organs located in the vicinity of the endoscope becomes too high, and there is a possibility that the tissues of the wall surfaces may be heated by the illumination light.

In view of such the present situation, the present inventors have considered that in the case where it becomes possible to change an illumination region only on a center portion of a screen, it becomes possible to observe in detail luminal organs located on the back side that have been difficult to observe in the past.

Here, although the Xe lamps used in the past are high luminance light sources used for various types of projecting apparatuses, such as a projector, they have the feature that Etendue (Etendue) represented by a product of a light emission area and a solid angle of light emission is very large. On the other hand, in the illumination of an endoscope, a light emission area and a radiation angle of illumination are small. As a result, Etendue also becomes small. Etendue is another expression of Helmholtz-Lagrange's conservation law, and, all of light with large Etendue cannot be put in small Etendue. That is, in an illumination system using a Xe lamp, in the case where a divergent angle of light emission is tried to be made smaller than the present situation, an amount of light becomes further small, illumination becomes dark, and, in addition, the utilization efficiency of the Xe lamp also lowers. Therefore, in the illumination system in the past, it is not possible to narrow the divergent angle of an illumination system, and an attempt to narrow a divergent angle of an illumination system has not been performed.

In view of the above-mentioned results of the studies, as a result of having performed further studies with an object to realize an endoscope light source capable of making an area of a region to be irradiated with illumination light changeable, the present inventors have conceived an endoscope light source, a control method of an endoscope light source, and an endoscope apparatus using such an endoscope light source, which will be mentioned in detail in the below.

### Embodiment

<With Regard to Entire Constitution of Endoscope Apparatus>

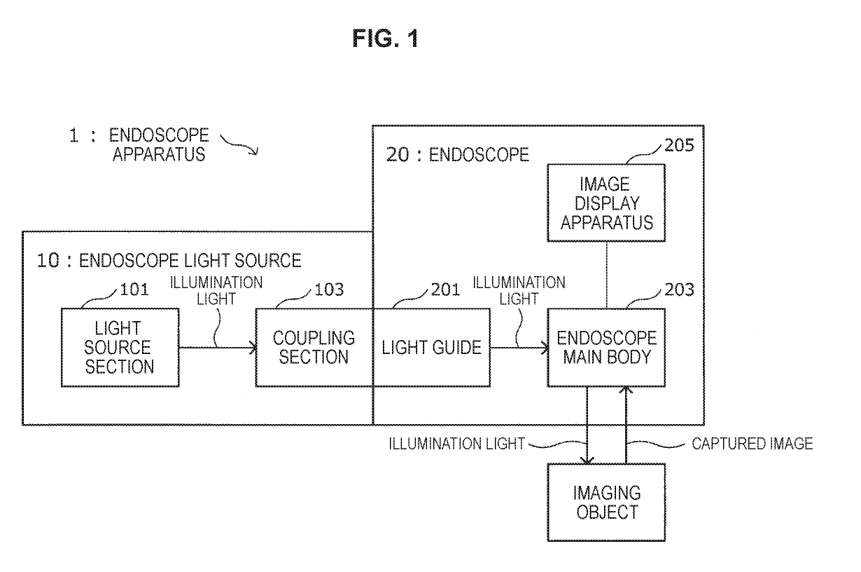

Hereinafter, first, with reference to FIG. 1, an entire constitution of an endoscope apparatus according to an embodiment of the present disclosure will be described. FIG. 1 is an explanatory diagram showing schematically an entire constitution of the endoscope apparatus according to the present embodiment.

An endoscope apparatus 1 according to the present embodiment includes an endoscope light source 10 and an endoscope 20 as shown in FIG. 1.

The endoscope light source 10 is an apparatus that emits light rays used as illumination light in the endoscope 20. As shown in FIG. 1, this endoscope light source 10 mainly includes a light source section 101 and a coupling section 103, and, is constituted so as to make it possible to make the area of a region irradiated with Illumination light changeable.

The light source section 101 includes at least one or more solid light sources, and, emits light from such a solid light source as illumination light. Moreover, in the case where the light source section 101 includes two or more solid light sources, the light source section 101 can also emit white light by mixing the colors of light from the respective solid light sources. A detailed constitution of this light source section 101 will be described again below. The illumination light emitted from the light source section 101 is guided to the coupling section 103 mentioned later.

The coupling section 103 is a section to be connected to a light guide that is disposed in the endoscope 20 and propagates a light flux (i.e., a light flux of illumination light) for connecting to the endoscope 20, and, is disposed to be able to be connected to such a light guide. The illumination light emitted from the light source section 101 is guided to the inside of the endoscope 20 through this coupling section 103. Moreover, in the endoscope light source 10 according to the present embodiment, as mentioned below in detail, this coupling section 103 is made to function as a center, whereby the incident angle of light rays entering the light guide is controlled. The detailed constitution of this coupling section 103 will be described below again.

The endoscope 20 is an apparatus a part of which is inserted into the inside of an examination object (imaging object), and that images the inside of the object and propagates an obtained captured image to a display apparatus such as a monitor. This endoscope 20 mainly includes, as shown in FIG. 1, a light guide 201, an endoscope main body 203, and an image display apparatus 205.

The light guide 201 is usually those in which a plurality of index guide type multi-mode optic fibers with a core diameter of 10 μm to 80 μm is bundled (bundled), and, propagates a light flux for connecting with the later-mentioned endoscope main body 203. The illumination light emitted from the endoscope light source 10 is propagated by this light guide 201, reaches the endoscope main body 203, and becomes to illuminate a prescribed region of an examination object being an imaging object via a bundle fiber disposed in the inside of the endoscope main body 203. Such the light guide 201 should not be limited specifically, and publicly-known light guides can be used.

The endoscope main body 203 is a section a part of which is inserted in the inside of an examination object (imaging object), and that images the inside of the examination object. As this endoscope main body 203, publicly-known endoscopes, such as a rigid endoscope and flexible endoscope for medical application and an endoscope for industry application, can be used.

The illumination light guided by the light guide 201 is propagated by the bundle fiber disposed in the endoscope main body 203, reaches the distal end portion of the endoscope main body 203, and illuminates a prescribed region of an imaging object. Moreover, at the distal end portion of the endoscope main body 203, an observation window for observing an imaging object is disposed, and an image of the imaging object through the observation window is propagated in the inside of the endoscope main body 203, and, is propagated up to a camera module (not shown) disposed on the other end of the endoscope main body. The image of the imaging object is made into digital data by various kinds of imaging elements disposed in the inside of a camera module, and, is output to a later-mentioned image display apparatus 205 at any time.

Moreover, a user of the endoscope 20 can acquire an enlarged image or a reduced image of a desired region of an imaging object by performing publicly-known operations, such as inserting and withdrawing the endoscope main body 203, driving a zoom optical system disposed in the endoscope main body 203, and actuating an electronic zoom function mounted in the endoscope 20.

The image display apparatus 205 is an apparatus that executes display control at the time of displaying a captured image with regard to an imaging object imaged by the endoscope main body 203 on a display screen of the image display apparatus 205 or various kinds of displays disposed on an external side of the image display apparatus 205. This image display apparatus 205 can be realized by, for example, information processing apparatuses, such as various kinds of computers including a CPU (Central Processing Unit), a ROM (Read Only Memory), a RAM (Random Access Memory), and so on. The image display apparatus 205 displays on a display screen by changing (namely, enlarging/reducing an image) the field angle of a captured image to be displayed on the display screen in accordance with the operation executed by the user of the endoscope 20.

In the above, while referring to FIG. 1, the entire constitution of the endoscope apparatus 1 according to the present embodiment has been described.

<With Regard to Constitution of Endoscope Light Source>

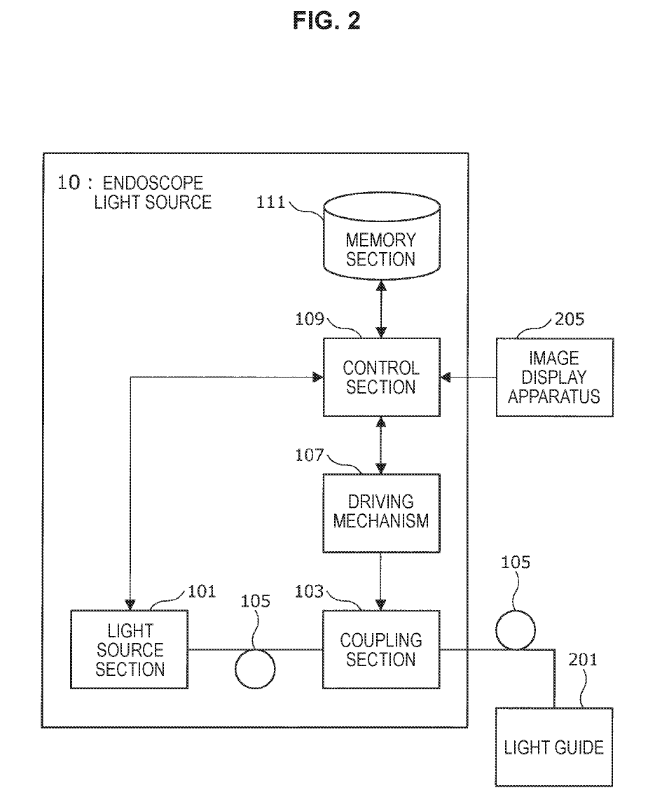

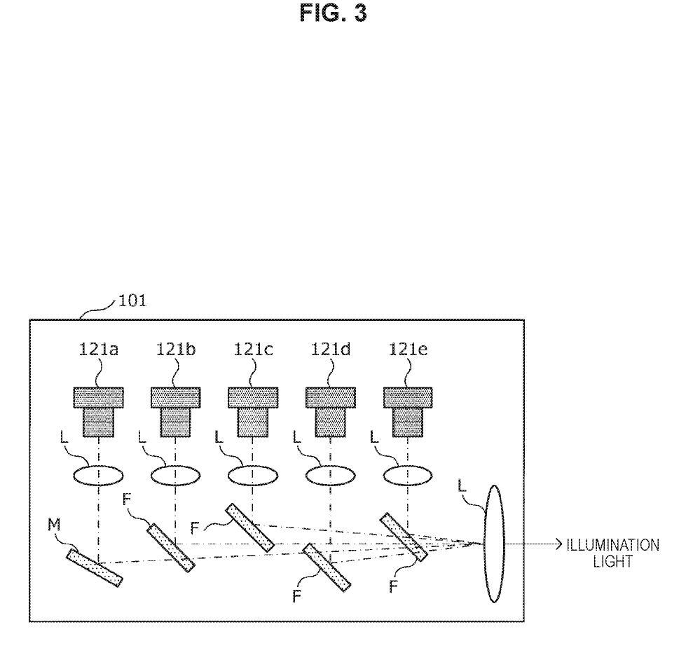

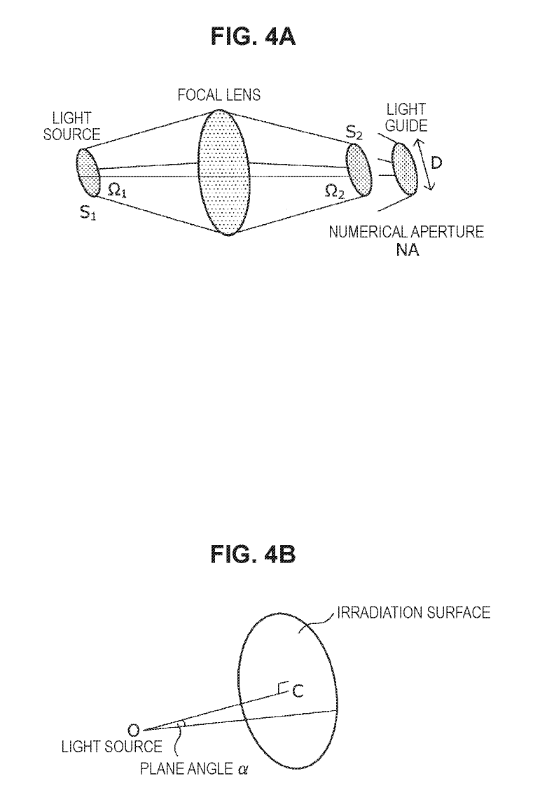

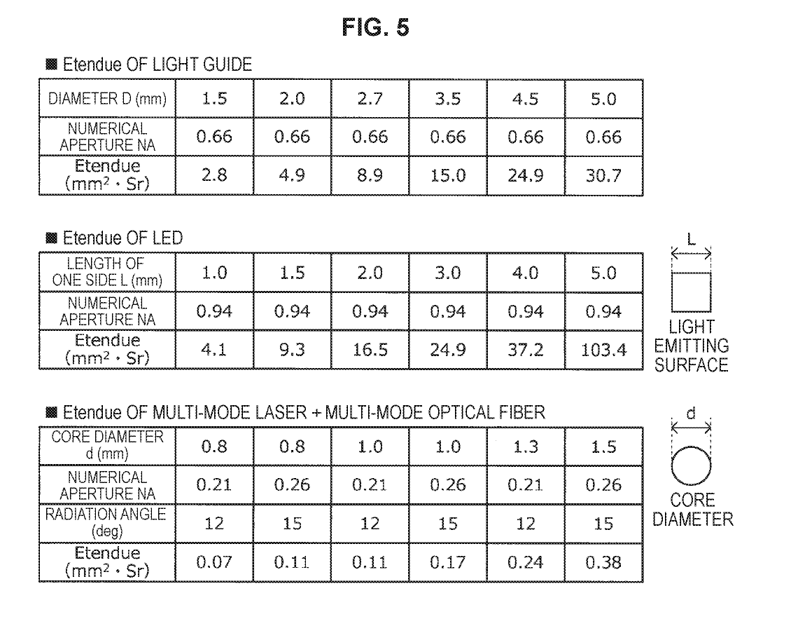

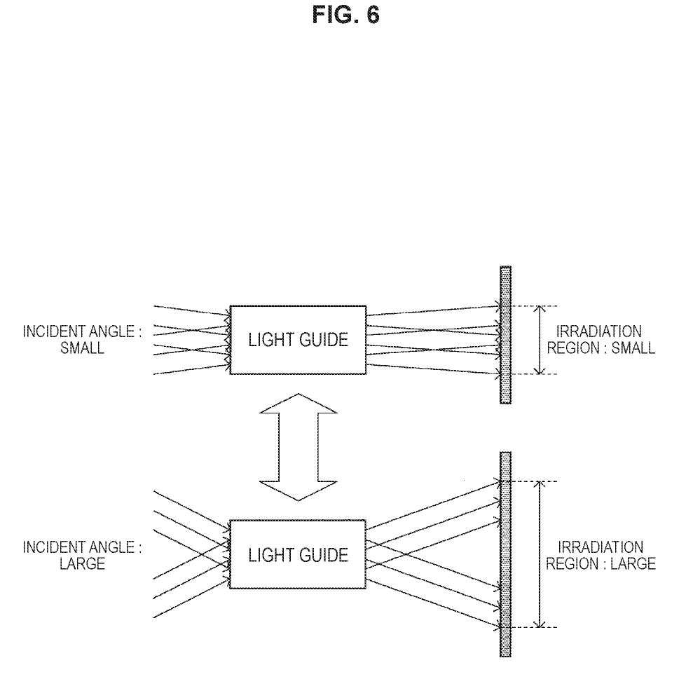

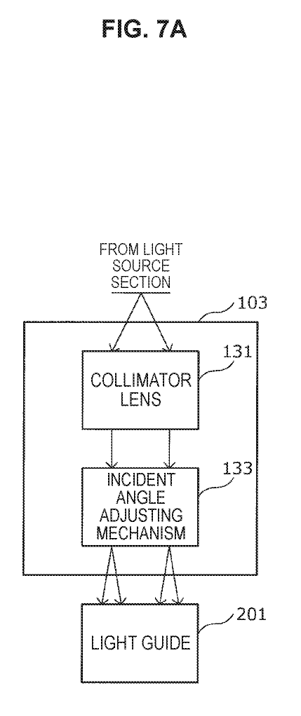

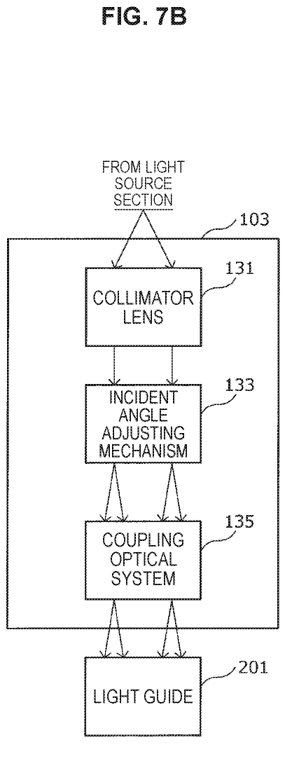

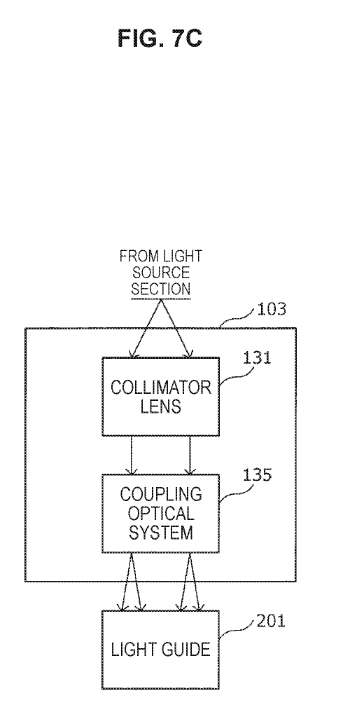

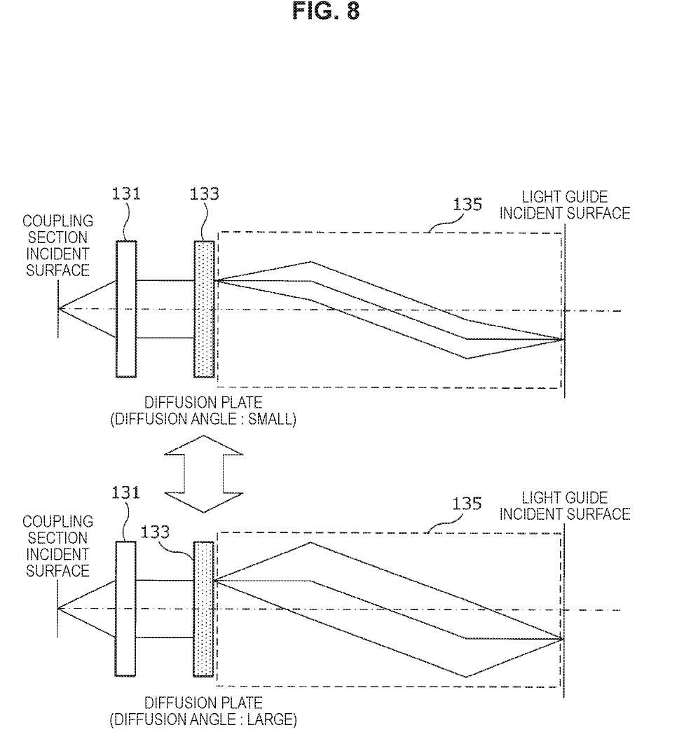

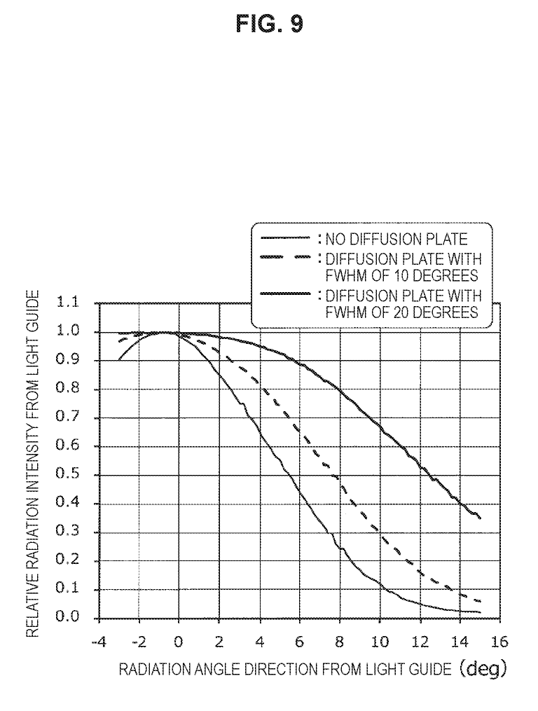

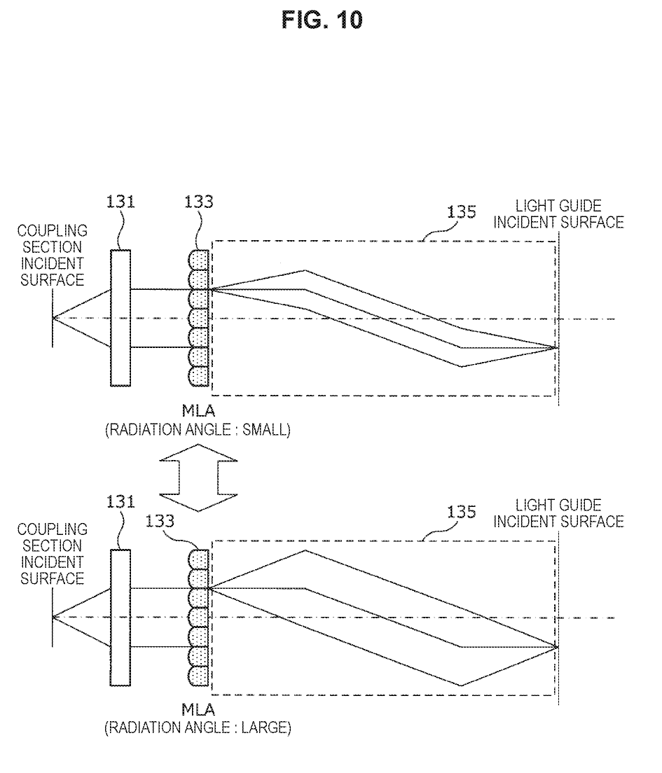

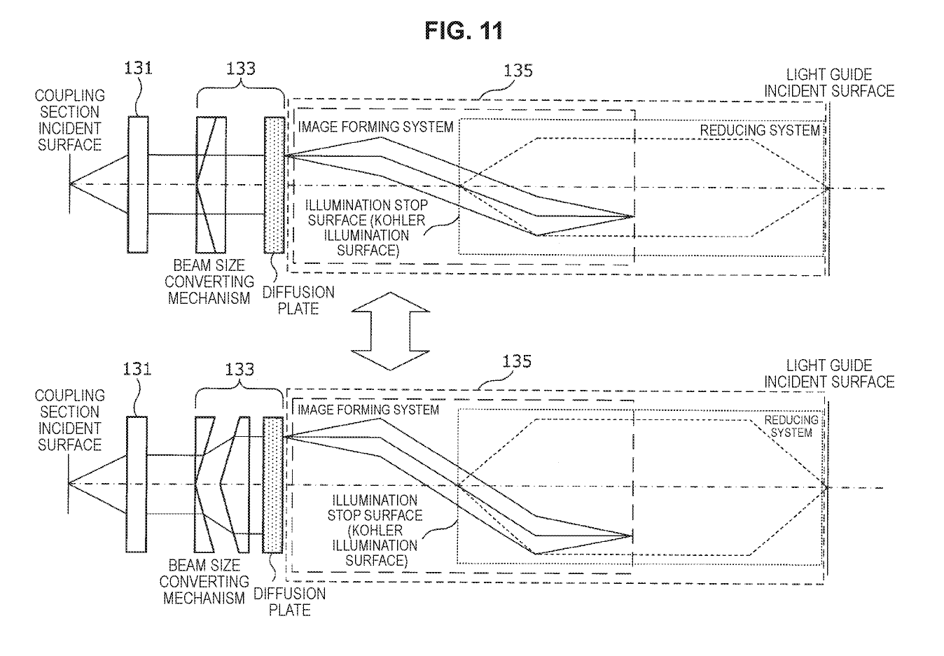

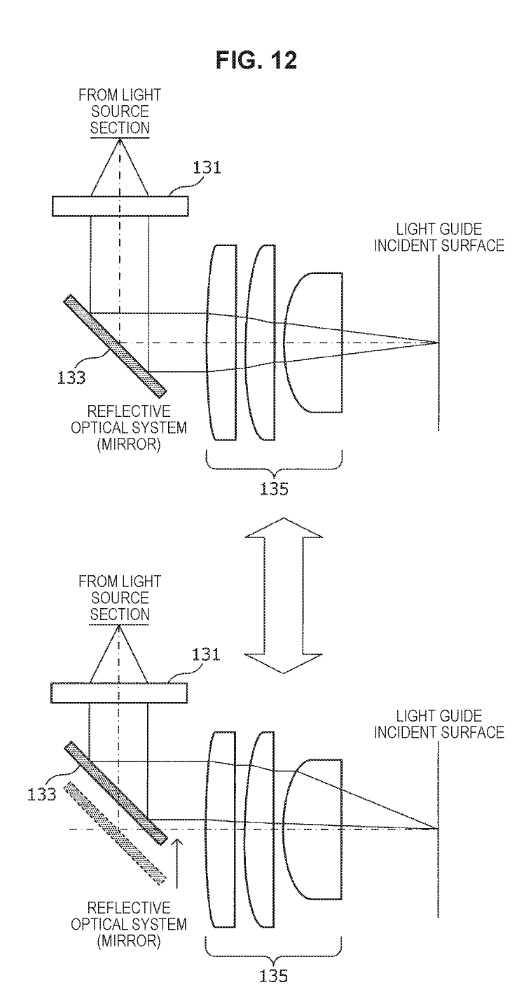

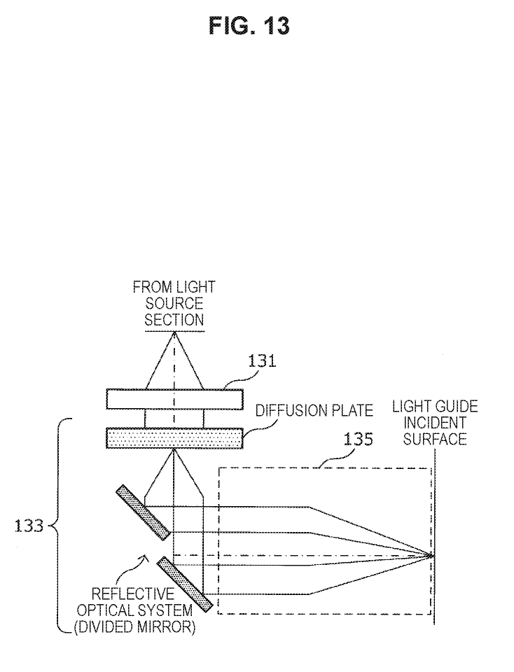

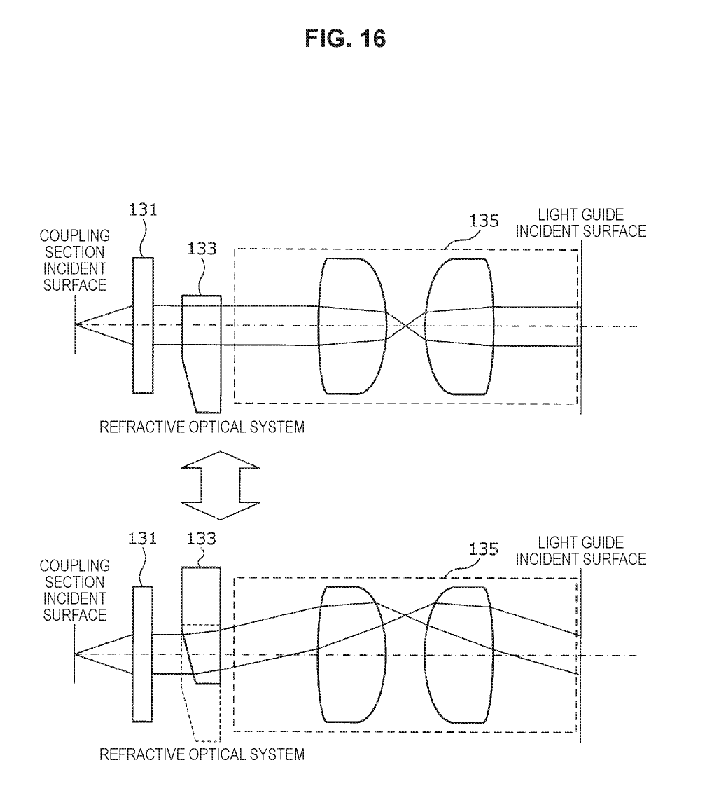

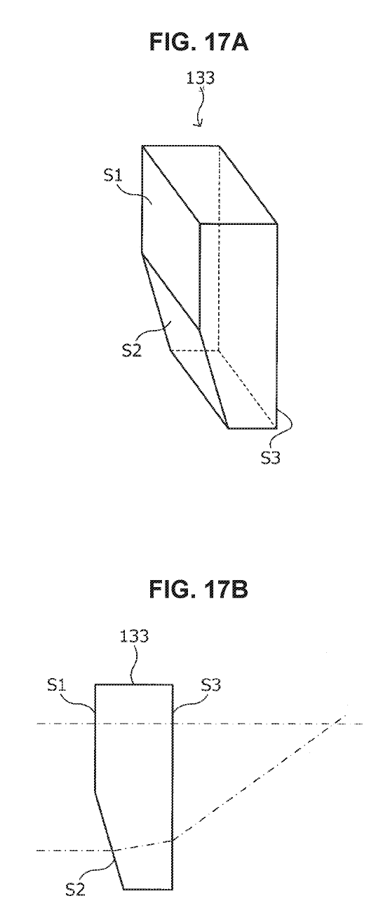

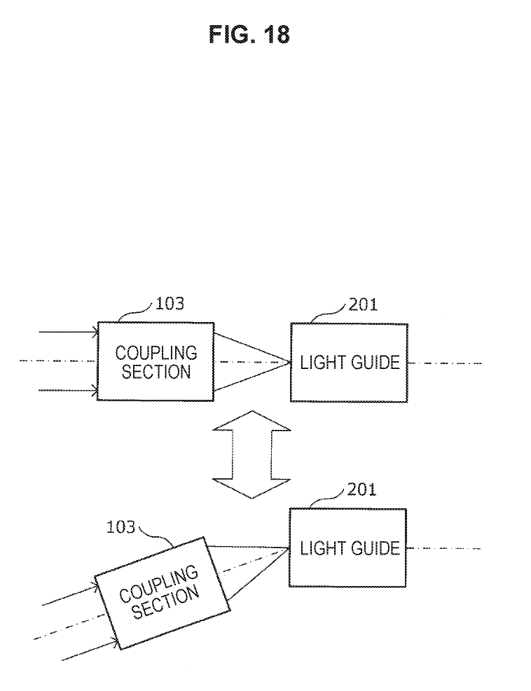

Next, a constitution of the endoscope light source 10 according to the present embodiment is described in detail while referring to FIG. 2 to FIG. 18. FIG. 2 is an explanatory diagram showing schematically a detailed constitution of the endoscope light source according to the present embodiment, and FIG. 3 is an explanatory illustration showing schematically one example of a light source section included in the endoscope light source according to the present embodiment. FIG. 4A to FIG. 5 are explanatory illustrations for describing Etendue, and FIG. 6 is an explanatory illustration for describing a controlling process of the incident angle of light rays relative to the light guide in the endoscope light source according to the present embodiment. FIG. 7A to FIG. 7C are explanatory diagrams showing schematically a constitution of the coupling section included in the endoscope light source according to the present embodiment. FIG. 8 is an explanatory illustration showing schematically the first concrete example of the coupling section according to the present embodiment, and FIG. 9 is a graph chart showing a relationship between the incident angle of light rays to the light guide and a radiation angle direction from the light guide. FIG. 10 is an explanatory illustration showing schematically the second concrete example of the coupling section according to the present embodiment, and FIG. 11 is an explanatory illustration showing schematically the third concrete example of the coupling section according to the present embodiment. FIG. 12 is an explanatory illustration showing schematically the fourth concrete example of the coupling section according to the present embodiment, and FIG. 13 to FIG. 15 are explanatory illustrations showing schematically the fifth concrete example of the coupling section according to the present embodiment. FIG. 16 to FIG. 17B are explanatory illustrations showing schematically the sixth concrete example of the coupling section according to the present embodiment, and FIG. 18 is an explanatory illustration showing schematically the seventh concrete example of the coupling section according to the present embodiment.

[With Regard to Entire Constitution]

First, the detailed entire constitution of the endoscope light source 10 according to the present embodiment is described while referring to FIG. 2. The endoscope light source 10 according to the present embodiment, in addition to the light source section 101 and the coupling section 103 that have been described while referring to FIG. 1, further includes a control section 109 as shown in FIG. 2, and preferably furthermore includes a multi-mode optical fiber 105, a driving mechanism 107, and a memory section 111.

The multi-mode optical fiber 105 is a multi-mode optical fiber having a core diameter of 10 μm or more, and, guides illumination light emitted from the light source section 101 to the coupling section 103. By connecting the light source section 101 and the coupling section 103 using the multi-mode optical fiber 105, it becomes possible to guide illumination light emitted from the light source section 101 to the coupling section 103 efficiently, and it becomes easy to handle the illumination light.

In this connection, in FIG. 1, the coupling section 103 and the light guide 201 are illustrated so as to be connected directly. However, as shown in FIG. 2, the coupling section 103 and the light guide 201 may be connected with the multi-mode optical fiber 105 having a core diameter of 10 μm or more. In this case, a light emitting side end surface of the multi-mode optical fiber 105 connected to the coupling section 103 functions as a virtual light source, and illumination light is guided to the light guide 201 by the coupling optical system that projects this virtual light source to the light guide.

The driving mechanism 107 is realized by publicly-known driving members, such as an actuator and a moving stage. The driving mechanism 107 sets the incident angle of light rays (i.e., light rays of illumination light) that enter the light guide 201 in the coupling section 103, so as to become a proper value by controlling an incident-angle adjusting mechanism disposed in the coupling section 103 as described in detail in the below under the control of the control section 109.

The control section 109 is realized by various kinds of IC chips including, for example, a CPU, a ROM, a RAM, and so on. The control section 109 is a processing section that totally controls the operation of the endoscope light source 10 according to the present embodiment, and manages, for example, an emitting process of illumination light from the light source section 101, a control process of the coupling section 103 by the driving mechanism 107, and so on. With this, it becomes possible for the control section 109 to control such that the incident angle of light rays that enter the light guide 201 in the coupling section 103, becomes changeable.

For more in details, the control section 109 makes the light source section 101 emit illumination light by outputting a predetermined control signal to the light source section 101. Moreover, upon acquisition of information that the field angle of a captured image to be displayed on a display screen has been changed, from the image display apparatus 205 of the endoscope 20, the control section 109 controls the driving mechanism 107 on the basis of such information so as to realize the irradiation region of illumination light corresponding to a change ratio of the field angle (a change ratio of the size of an image). Moreover, in addition to such control for the irradiation region, the control section 109 may control the light source section 101 so as to emit a proper amount of illumination light, if necessary. That is, when the irradiation region of illumination light has been changed, in the case where an amount of illumination light is too much in an irradiation region after the changing (i.e., in the case of too bright), the control section 109 controls the light source section 101 to lower the intensity of illumination light emitted from the light source section 101 so as to become a proper amount of light. Moreover, when the irradiation region of illumination light has been changed, in the case where an amount of illumination light is too small in an irradiation region after the changing (i.e., in the case of too dark), the control section 109 controls the light source section 101 to increase the intensity of illumination light emitted from the light source section 101 so as to become a proper amount of light.

Here, with regard to whether an amount of illumination light on an irradiation region is proper or not, it is possible to determine whether or not it is proper, by setting a predetermined threshold beforehand with regard to an amount of illumination light and by determining whether an amount of illumination light on an irradiation region after the changing is larger or smaller than the threshold set beforehand. Moreover, with regard to an area of an illumination region and a proper amount of illumination light, it is possible to set properly by making the value of a proper area of an illumination region corresponding to a change ratio of a size of an image and the value of a proper amount of light corresponding to an area of an irradiation region into a data base in a format, for example, like a look-up table and by referring to such a data base.

In this connection, at the time of executing various kinds of control processes, it is possible for the control section 109 to use various kinds of parameters, a data base, various kinds of programs, etc. that are stored in the memory section 111. Moreover, the control section 109 may control the incident angle of light rays that enter the light guide 201 in the coupling section 103, in accordance with various kinds of user's operations executed by a user of the endoscope 20 who has confirmed the image display apparatus 205.

The memory section 111 is realized by, for example, a ROM, a RAM, a storage device, and so on. In the memory section 111, various kinds of parameters, a data base, various kinds of programs, etc. are stored that are able to be referred when the control section 109 executes various kinds of control processes. Moreover, in this memory section 111, temporary data, various kinds of history information, etc. may be stored that are created when the control section 109 executes various kinds of control processes. For this memory section 111, it is possible for the control section 109 to execute reading/writing processes of data freely.

In the above, the detailed entire constitution of the endoscope light source 10 according to the present embodiment has been described while referring to FIG. 2.

[With Regard to Constitution of Light Source Section 101]

Next, one example of a constitution of the light source section 101 included in the endoscope light source 10 according to the present embodiment is described in detail while referring to FIG. 3 to FIG. 5.

It is preferable that, for example as shown in FIG. 3, the light source section 101 according to the present embodiment includes a plurality of solid light sources 121a, 121b, 121c, 121d, 121e . . . (hereinafter, collectively referred to also as a solid light source 121). From each of the solid light sources 121, light of a prescribed wavelength is emitted. Here, a combination of the wavelengths of light emitted from each of the solid light sources 121 is not limited specifically. However, as a result of mixing the colors of light emitted from each of the solid light sources 121, a combination capable of obtaining white light is preferable. As a combination of such wavelengths, it is preferable that, for example, any one of the solid light sources 121a to 121e emits red light, any one of the solid light sources 121a to 121e emits a green light, and any one of the solid light sources 121a to 121e emits blue light. Moreover, any one of the solid light sources 121a to 121e may emit purple light, and any one of the solid light sources 121a to 121e may emit infrared light.

The propagating direction of light emitted from each of the solid light sources 121 is controlled by a lens L, a mirror M, and an optical filter F disposed at a stage following each of the solid light sources 121, and color mixture is finally performed by a lens L disposed at a stage following the mirror M and the optical filter F. Here, the mirror M has an optical property to reflect light emitted from the solid light source 121a, and each of the optical filters F has an optical property that reflects light emitted from a solid light source 121 disposed at an upstream side of each of the optical filters F and allows light having wavelength bands other than it to pass through. The light after having been subjected to color mixture is emitted to the outside of the light source section 101 as illumination light.

Here, while referring to FIG. 4A to FIG. 5, the relationship between the Etendue of the solid light source 121 and the Etendue of the light guide 201 is described concretely.

As mentioned above, Etendue is another representation of Helmholtz-Lagrange's conservation law, and, is expressed by the product of a light emitting area and the solid angle of light rays. Now, as shown in FIG. 4A, it is assumed that the light emitting area of a light source and the solid angle of light emitted from the light source are denoted as S₁ and Ω₁ respectively, and the area of an incident surface of the light guide 201 and the solid angle of light that enters the incident surface, are denoted as S₂ and Ω₂ respectively. At this time, since the value of Etendue is conserved in the optical system to be noticed, Formula 101 shown below is established. Here, the unit of the Etendue becomes [mm²·sr] (square millimeter·steradian) in the case of using an SI unit system.

Moreover, in the case where light is radiated in rotation symmetry relative to an optical axis, a solid angle [unit: sr] can be expressed by Formula 103 shown below in the case of using a plane angle α [unit: rad] as shown in FIG. 4B, and the numerical aperture NA of a light guide can be expressed by Formula 105 shown below by using the plane angle α. Therefore, Formula 101 that gives the value of Etendue can be expressed by Formula 107 shown below by using Formula 103 and Formula 105 shown below. Here, in Formula 107 shown below, D represents a diameter of a light guide.

$$\begin{matrix}[ Math . 1 ] & \\ {S}_{1} \cdot {Ω}_{1} = {S}_{2} \cdot {Ω}_{2} & ( Formula 101 ) \\ Ω = 2 π ( 1 - cos α ) & ( Formula 103 ) \\ NA = sin α & ( Formula 105 ) \\ {S}_{1} \cdot {Ω}_{1} = {S}_{2} \cdot {Ω}_{2} = {π ( \frac{D}{2} )}^{2} \cdot 2 π ( 1 - \sqrt{1 - {( NA )}^{2}} ) & ( Formula 107 )\end{matrix}$$

If generalizing, Etendue (hereinafter, its value is denoted as E) can be expressed by Formula 109 shown below by using the radiation angle distribution I (θ, φ) (θ, φ: a radiation angle of light rays) of the intensity of light rays emitted from a light source. Here, it is assumed that a light source to be noticed is a Lambertian (Lambertian) light source, the radiation angle distribution I (θ, φ) of the intensity can be expressed by Formula 111 shown below by using the intensity I₀. In that case, Etendue becomes like Formulas 113 shown below. On the other hand, since the relation of Formula 115 shown below is established, the Etendue of the Lambertian light source becomes smaller than that of a light source having no radiation angle distribution.

$$\begin{matrix}[ Math . 2 ] & \\ E = S \cdot Ω = S \int I ( θ , φ ) d Ω & ( Formula 109 ) \\ I ( θ , φ ) = {I}_{0} \cdot cos θ & ( Formula 111 ) \\ E = S \cdot {I}_{0} \cdot π ( 1 - {cos}^{2} θ ) = {SI}_{0} {π ( NA )}^{2} & ( Formula 113 ) \\ {π ( NA )}^{2} < 2 π \sqrt{1 - {( NA )}^{2}} = 2 π ( \frac{{( NA )}^{2}}{2} + \frac{{( NA )}^{4}}{8} + \frac{{( NA )}^{6}}{16} + … ) & ( Formula 115 )\end{matrix}$$

Here, in the case where the Etendue of a light guide with a common diameter D and numerical aperture NA is calculated on the presupposition that the radiation angle distribution I (θ, φ) of intensity is uniform at I₀, it becomes like a table shown at the uppermost stage in FIG. 5. Therefore, with regard to the light from a light source with the Etendue larger than the Etendue shown in the uppermost stage in FIG. 5, all of it cannot be coupled to a light guide. On the other hand, with regard to the light from a light source with the Etendue smaller than the Etendue shown in the uppermost stage in FIG. 5, all of it can be coupled to a light guide.

Therefore, it is preferable that the solid light sources 121 used in the light source section 101 according to the present embodiment is a light source having the Etendue equal to or less than the Etendue of the light guide 201. By using such a solid light source, it becomes possible to use all of light emitted from the solid light source, and it is possible to improve the utilization efficiency of the light source.

In such a viewpoint, it turns out that, since a light emitting point is very small, a light source preferable as a solid light source is a laser light source (for example, semiconductor laser light source) that can emit parallel light (that is, a solid angle becomes almost zero) easily by an optical system. Moreover, it is also possible to use a laser excitation phosphor light source in which such a laser light source is used as an excitation light source for a phosphor.

Moreover, although the development of a light emitting diode (Light Emitting Diode: LED) element is also active in recent years, since light emission in the LED elements is surface light emission, a light emitting region becomes large. Accordingly, the value of Etendue becomes larger than that of the laser light source. However, depending on its performance, it is possible to use it as the solid light source according to the present embodiment.

Now, in the case where the Etendue of a general surface light emission type square LED (a length of one side: L) is calculated on the presupposition that the radiation angle distribution of intensity satisfies the above-described Formula 111, it becomes a value like a table shown at a middle stage in FIG. 5. By using such a table, in the case of calculating the upper limit value of a light emitting area that can introduce all of light into a light guide, the value becomes 5.8 mm² (L≈2.4 mm).

Moreover, in the single mode laser, the light emitting area is very small, and the Etendue also becomes a very small value. However, in the case of realizing high output, it is required to use a plurality of multi-mode lasers. Accordingly, it is difficult to generalize the value of Etendue. Then, it is assumed a case where the laser light from a multi-mode laser is coupled to a general multi-mode optical fiber with a certain core diameter d and numerical aperture NA, and Etendue has been calculated by making this optical fiber as a virtual light source. The obtained results are shown in a table at the lowermost stage in FIG. 5. As is clear from this table, it turns out that it is possible to couple the light of the laser light source introduced into the multi-mode optical fiber to even a small diameter light guide with a diameter of 1.5 mm at an efficiency of 100%.

In the above, while referring to FIG. 3 to FIG. 5, one example of the light source section 101 according to the present embodiment has been described in detail. In this connection, the constitution of the light source section 101 shown in FIG. 3 is merely one example, and the constitution of the light source section 101 according to the present embodiment should not be limited to one shown in FIG. 3.

[With Regard to Constitution of Coupling Section 103]

Next, while referring to FIG. 6 to FIG. 18, a constitution of the coupling section 103 included in the endoscope light source 10 according to the present embodiment is described in detail.

As a result of having studied earnestly an endoscope light source capable of making the area of a region irradiated with illumination light changeable, the present inventors have obtained the knowledge that it is possible to control the radiation angle of light rays emitted from a light guide by changing the incident angle (angle formed by incident light rays relative to the optical axis of a light guide) of light rays that enter the light guide.

Namely, as shown schematically in FIG. 6, in the case where light rays enter at a small incident angle relatively to a light guide, the radiation angle of the light rays emitted from the light guide becomes a small value (at the upper stage in FIG. 6). In the case where light rays enter at a large incident angle relatively to the light guide, the radiation angle of the light rays emitted from the light guide becomes a large value (at the lower stage in FIG. 6). The reasons are that a general light guide is those in which a plurality of index guide type multi-mode optical fibers with a core diameter of about 10 μm to 80 μm are bundled (bundled) and that the optical fiber has the characteristics to radiate light rays from an emitting end surface while keeping the angle of the light rays having entered an incident end surface. However, in the optical fiber, although the incident angle of light rays is preserved, the incident position of the light rays is not preserved. Accordingly, the light rays having entered at a certain incident angle become ring-shaped light rays while keeping the angle, and then, are radiated from the emitting end surface.

As shown schematically at an upper stage in FIG. 6, with this phenomenon, by making the incident angle of light rays to the light guide relatively small, the radiation angle of the light rays from the light guide becomes small. As a result, it becomes possible to narrow the irradiation region of the light rays radiated from the light guide to small. On the contrary, as shown schematically at a lower stage in FIG. 6, by making the incident angle of light rays to the light guide relatively large, the radiation angle of the light rays from the light guide becomes large. As a result, it becomes possible to greatly expand the irradiation region of the light rays radiated from the light guide.

In the coupling section 103 according to the present embodiment, the incident angle of light rays to the light guide is controlled as described in the above, thereby controlling the radiation angle of the light rays introduced to the light guide and making the area of a region irradiated with illumination light changeable.

Here, the coupling section 103 may control the incident angle of light rays that enter a light guide, to two kinds of incident angles, for example, an incident angle close to parallel light and an incident angle close to the numerical aperture NA of the light guide, or, may control the incident angles from an incident angle close to parallel light to an incident angle close to the numerical aperture NA of the light guide to multi stages.

It is preferable that the coupling section 103 having such a function includes at least a collimator lens 131 and an incident angle adjusting mechanism 133 as shown in FIG. 7A. The collimator lens 131 is an optical element that makes illumination light that has entered the coupling section 103 from the light source section 101, to parallel light. Moreover, the incident angle adjusting mechanism 133 is a mechanism that adjusts the incident angle of illumination light to the light guide as having described while referring to FIG. 6. As the driving mechanism 107 shown in FIG. 2 functions, the state of the incident angle adjusting mechanism 133 changes so as to change, for example, the beam size or divergent angle of the light having entered the coupling section 103, whereby the incident angle adjusting mechanism 133 changes the incident angle of the illumination light to the light guide. A concrete example of this incident angle adjusting mechanism 133 will be described again in the below.

Moreover, it is preferable that the coupling section 103 according to the present embodiment further includes a coupling optical system 135 at the stage following the incident angle adjusting mechanism 133 as shown in FIG. 7B. The coupling optical system 135 is an optical system that couples light rays whose incident angle to the light guide has been controlled, to the light guide 201 of the endoscope 20. By providing such an optical system, it becomes possible to couple light rays whose incident angle to the light guide 201 has been controlled, to the light guide 201 more certainly. As such an optical system, it is possible to apply a publicly-known optical system such as a fixed magnification optical system as long as it does not change the controlled incident angle of the illumination light.

Moreover, as shown in FIG. 7C, in the coupling section 103 according to the present embodiment, the coupling optical system 135 may also have the function of the incident angle adjusting mechanism 133. That is, by changing the magnification of the coupling optical system 135, it becomes possible to change the beam size of illumination light on the incident surface of the light guide 201. Owing to such a change of the beam size, since the incident angle of illumination light on the incident surface of the light guide 201 changes, it becomes possible to realize the control of an illumination region as having described with reference to FIG. 6.

In the case of performing the control of the area of an illumination region and narrowing the illumination region in this way, an amount of illumination light having dispersed to a wide area before the changing, is concentrated to the narrowed illumination region after the changing. As a result, it becomes possible to make the illumination region brighter, and, in addition, it becomes possible to use the illumination light more efficiently.

### First Concrete Example of Coupling Section 103

The first concrete example of the coupling section 103 having the above-described functions is described while referring to FIG. 8 and FIG. 9. In the first concrete example of the coupling section 103 shown in FIG. 8, a diffusion plate is used as the incident angle adjusting mechanism 133. By using the diffusion plate as the incident angle adjusting mechanism 133, it is possible to change the divergent angle of light rays (i.e., illumination light) that enter the diffusion plate, and, with this, it is possible to change the incident angle of light rays to the light guide 201.

Namely, in the coupling section 103 in the first concrete example, the diffusion plate is disposed as the incident angle adjusting mechanism 133 at the stage following the collimator lens 131, and the fixed magnification optical system as one example of the coupling optical system 135 is disposed at the stage following the diffusion plate. In this case, as shown at an upper stage in FIG. 8, in the case where a diffusion plate with a small diffusion angle is disposed on the optical path, the incident angle of illumination light on the incident surface of the light guide 201 becomes a relatively small angle, and the irradiation region of the illumination light becomes narrow relatively. On the other hand, as shown at a lower stage in FIG. 8, in the case where a diffusion plate with a large diffusion angle is disposed on the optical path, the incident angle of the illumination light on the incident surface of the light guide 201 becomes a relatively large angle, and the irradiation region of the illumination light becomes wide relatively.

FIG. 9 shows the result of having measured the radiation angle of light rays radiated from the emitting end of a general light guide with regard to three cases of a case of not disposing a diffusion plate, a case of having disposed a diffusion plate with a diffusion angle of 10 degrees (full width at half maximum), and a case of having disposed a diffusion plate with a diffusion angle of 20 degrees (full width at half maximum). As shown in FIG. 9, the value of a radiation angle at which an amount of light is lowered up to 50% is about 5.5 degrees in the case of not disposing a diffusion plate, about 7.5 degrees in the case of having disposed a diffusion plate with a diffusion angle of 10 degrees, and about 12.5 degrees in the case of having disposed a diffusion plate with a diffusion angle of 20 degrees. As is clear from this result, by controlling the divergent angle of illumination light that enters the light guide 201, by utilizing the diffusion plate, it becomes possible to change the irradiation region of the illumination light.

Therefore, in the coupling section 103, by preparing a plurality of diffusion plates different in diffusion angle, and by replacing the diffusion plate to be disposed on the optical path with the driving mechanism 107, it becomes possible to realize the above functions. In this connection, not only by replacing the plurality of diffusion plates different in diffusion angle, but also, by increasing or decreasing the number of diffusion plates to be disposed on the optical path, it is possible to obtain the effects similar to the above.

### Second Concrete Example of Coupling Section 103

Next, the second concrete example of the coupling section 103 is described while referring to FIG. 10. In the first concrete example, as the incident angle adjusting mechanism 133, the diffusion plate is disposed. However, in the second concrete example, as the incident angle adjusting mechanism 133, a multi lens array (Multi Lens Array: MLA) in which a plurality of lenses is arranged in an array form, is disposed. By changing the focal length of the multi lens array to be disposed on the optical path, it is possible to change the divergent angle of light rays (i.e., illumination light) that enter the multi lens array. With this, it is possible to change the incident angle of light rays to the light guide 201.

That is, in the coupling section 103 in the second concrete example, the multi lens array is disposed at the stage following the collimator lens 131 as the incident angle adjusting mechanism 133, and a fixed magnification optical system is disposed at the stage following the multi lens array as an example of the coupling optical system 135. As shown at an upper stage in FIG. 10, in the case where a multi lens array with a long focal length is disposed on the optical path, the incident angle of illumination light on the incident surface of the light guide 201 becomes a relatively small angle, and the irradiation region of illumination light becomes narrow relatively. On the other hand, as shown at a lower stage in FIG. 10, in the case where a multi lens array with a short focal length is disposed on the optical path, the incident angle of illumination light on the incident surface of the light guide 201 becomes a relatively large angle, and the irradiation region of illumination light becomes wide relatively.

Therefore, in the coupling section 103, by preparing a plurality of multi lens arrays different in focal length, and by replacing the multi lens array to be disposed on the optical path with the driving mechanism 107, it becomes possible to realize the function like the above. In this connection, not only by replacing the plurality of multi lens arrays different in focal length, but also, by increasing or decreasing the number of multi lens arrays to be disposed on the optical path, it is possible to obtain the effects similar to the above.

### Third Concrete Example of Coupling Section 103

Next, the third concrete example of the coupling section 103 is described while referring to FIG. 11. In the third concrete example, as the incident angle adjusting mechanism 133, a beam size converting mechanism capable of being separated into a lens with a conical surface and a lens with a concave surface corresponding to the conical surface and a diffusion plate are disposed. This beam size converting mechanism can convert the beam size of entering illumination light by separating the two lenses and changing the distance between the two lenses. That is, in the case where the two lenses are united, the beam size of the entering illumination light is maintained as it was in the state of having entered. On the other hand, in the case where the lens with the conical surface is separated away, it becomes possible to convert the beam size of the entering illumination light to a large size. Therefore, it can be said that this beam size converting mechanism is an optical element capable of creating a virtual light surface optically. By making illumination light having passed through the beam size converting mechanism further diffuse by a diffusion plate, and by coupling it to the incident surface of the light guide 201 by the coupling optical system (in this case, the coupling optical system includes a fixed magnification optical system and a reducing optical system) disposed at the stage following the diffusion plate, it is possible to make the incident angle of light rays to the light guide 201 change.

That is, in the coupling section 103 in the third concrete example, as shown at an upper stage in FIG. 11, in the case where the beam size converting mechanism is not separated to two pieces, the incident angle of illumination light on the incident surface of the light guide 201 becomes a relatively small angle, and the irradiation region of illumination light becomes narrow relatively. On the other hand, as shown at a lower stage in FIG. 11, in the case where the beam size converting mechanism is separated into two pieces, the incident angle of illumination light on the incident surface of the light guide 201 becomes a relatively large angle, and the irradiation region of illumination light becomes wide relatively.

Therefore, in the coupling section 103, by controlling the separation state of the beam size converting mechanism by the driving mechanism 107, it becomes possible to realize the function like the above.

### Fourth Concrete Example of Coupling Section 103

Next, the fourth concrete example of the coupling section 103 is described while referring to FIG. 12. In the fourth concrete example, as the incident angle adjusting mechanism 133, a reflective optical system such as a mirror is disposed, and by controlling an incident position to the coupling optical system 135, it becomes possible to change the incident angle of light rays to the light guide 201.

Namely, as shown at an upper stage in FIG. 12, by controlling the position of the reflective optical system so as to control to make illumination light from the light source section 101 enter near the optical axis of the coupling optical system 135, the incident angle of the illumination light on the incident surface of the light guide 201 becomes a relatively small angle, and the irradiation region of the illumination light becomes narrow relatively. On the other hand, as shown at a lower stage in FIG. 12, by controlling the position of the reflective optical system so as to control to make illumination light from the light source section 101 enter a position away from the optical axis of the coupling optical system 135, the incident angle of the illumination light on the incident surface of the light guide 201 becomes a relatively large angle, and the irradiation region of the illumination light becomes wide relatively. In this connection, in the case shown at a lower stage in FIG. 12, illumination light enters the light guide 201 from a certain one direction. However, in the light guide 201 including a plurality of optical fibers, as described earlier, although an incident angle is preserved, an incident position is not preserved. Accordingly, illumination light having entered from one direction becomes to be diffracted over the entire circumference, whereby it becomes possible to illuminate the entirety of a desired region.

Therefore, in the coupling section 103, by controlling the position of the reflective optical system such as a mirror by the driving mechanism 107, it becomes possible to realize the function like the above.

### Fifth Concrete Example of Coupling Section 103

Next, the fifth concrete example of the coupling section 103 is described while referring to FIG. 13 to FIG. 15. In the fourth concrete example, as a control method of a mirror, only a simple transverse movement as shown in FIG. 12 has been described. However, by executing control, such as separating mirrors and then moving both the mirrors in the respective directions reverse to each other, or moving one of them in the radial direction, it becomes possible to control the incident angle variously similarly to the fourth concrete example. In the below, a concrete example where such mirrors are divided is described briefly.

In the present concrete example, as shown schematically in FIG. 13, as the incident angle adjusting mechanism 133, a reflective optical system such as divided mirrors (hereinafter, also merely referred to as “divided mirrors”) is disposed. By moving at least any one of such divided mirrors, the incident angle of illumination light to the coupling optical system 135 is controlled, whereby the incident angle of light rays to the light guide 201 is changed.

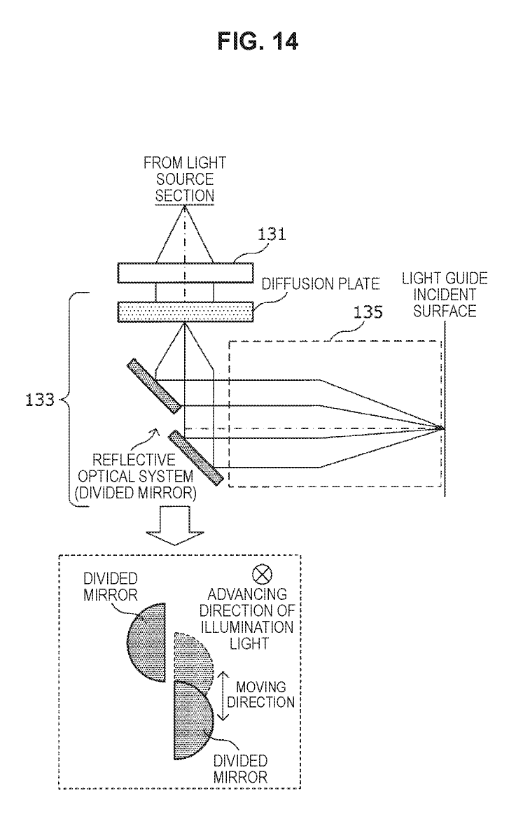

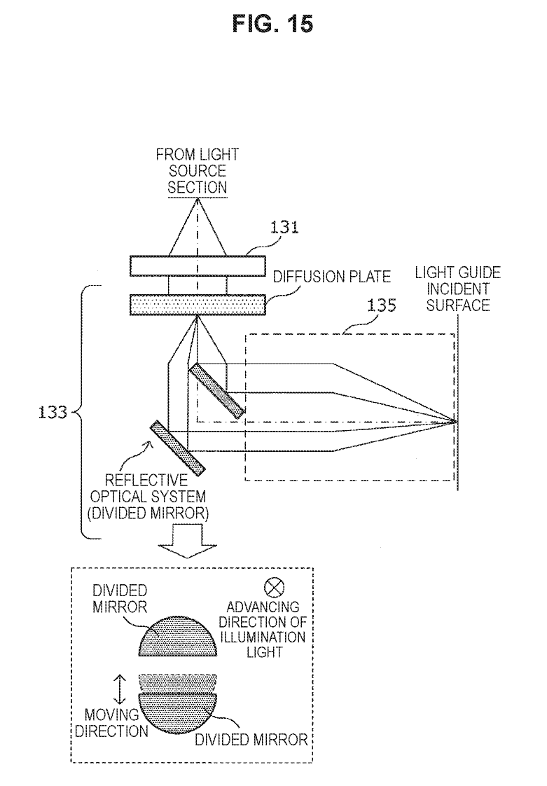

In concreter terms, the reflective optical system which has been a single mirror in the fourth concrete example may be divided into two mirrors in which one of them is located on the sheet surface front side of a flat surface parallel to the sheet surface and the other one is located on the sheet surface back side so as to form a configuration shown in FIG. 14. Alternatively, the reflective optical system which has been a single mirror in the fourth concrete example may be divided into two mirrors in which one of them is located on the sheet surface upper side of a flat surface vertical to the sheet surface and the other one is located on the sheet surface lower side so as to form a configuration shown in FIG. 15.

In addition to the above, in the example shown in FIG. 14, by moving any one of the divided mirrors in the radial direction (i.e., in the vertical direction on the sheet surface), it becomes possible to change the incident angle of illumination light on the incident surface of the light guide 201. Similarly, in the example shown in FIG. 15, by moving at least any one of the divided mirrors (for example, the position of the divided mirror on the upper side is fixed, and the divided mirror on the lower side is moved, or, the divided mirror on the upper side is moved downward, and the divided mirror on the lower side is moved upward, and the like), it becomes possible to change the incident angle of illumination light on the incident surface of the light guide 201.

Therefore, in the coupling section 103, by controlling the position of the reflective optical systems, such as divided mirrors, with the driving mechanism 107, it becomes possible to realize the function like the above.

### Sixth Concrete Example of Coupling Section 103

Next, the sixth concrete example of the coupling section 103 is described while referring to FIG. 16 and FIG. 17. In the sixth concrete example, as shown schematically in FIG. 16, as the incident angle adjusting mechanism 133, a refractive optical system, such as a structure prism, is disposed. Accordingly, by controlling the incident angle of illumination light to the coupling optical system 135, it becomes possible to change the incident angle of light rays to the light guide 201.

One example of a structure of the structure prism is shown in FIG. 17A and FIG. 17B. The structure prism capable of being used as the incident angle adjusting mechanism 133 includes optically transmitting surfaces S1, S2, and S3 as shown in FIG. 17A and FIG. 17B. The optically transmitting surface S1 and the optically transmitting surface S3 are parallel to each other. Moreover, the optically transmitting surface S2 and the optically transmitting surface S3 are not parallel to each other, and the optically transmitting surface S2 forms an inclined surface with a predetermined angle. As shown in FIG. 17B, the optically transmitting surface S1 and the optically transmitting surface S3 are made vertical to the optical axis of the optical system in which this structure prism is disposed, the optical axis of light that enters the optically transmitting surface S1 and is emitted from the optically transmitting surface S3, is parallel to the optical axis of the optical system, and the advancing direction of the light does not change. However, since he optically transmitting surface S2 is inclined relative to the optical axis of the optical system in which this structure prism is disposed, the optical axis of light that enters the optically transmitting surface S2 and is emitted from the optically transmitting surface S3, has an angle corresponding to the inclination angle of the optically transmitting surface S2 due to the effect of refraction.

As shown at an upper stage in FIG. 16, by utilizing such a structure prism, by controlling the position of a refractive optical system (structure prism), and by controlling illumination light from the light source section 101 so as to enter in almost parallel to the optical axis of the coupling optical system 135, the incident angle of the illumination light on the incident surface of the light guide 201 becomes a relatively small angle, and the irradiation region of the illumination light becomes narrow relatively. On the other hand, as shown at a lower stage in FIG. 16, by controlling the position of the refractive optical system, and by controlling illumination light from the light source section 101 so as to enter with an angle to the optical axis of the coupling optical system 135, the incident angle of the illumination light on the incident surface of the light guide 201 becomes a relatively large angle, and the irradiation region of the illumination light becomes wide relatively.

In this connection, in the case shown at the lower stage in FIG. 16, illumination light enters the light guide 201 from a certain one direction. However, in the light guide 201 including a plurality of optical fibers, as described earlier, although an incident angle is preserved, an incident position is not preserved. Accordingly, illumination light having entered from one direction becomes to be diffracted over the entire circumference, whereby it becomes possible to illuminate the entirety of a desired region.

Therefore, in the coupling section 103, by controlling the position of the refractive optical system such as a structure mirror with the driving mechanism 107, it becomes possible to realize the function like the above.

In this connection, in the sixth concrete example, the refractive optical system such as a structure mirror is disposed between the collimator lens 131 and the coupling optical system 135. However, even if the refractive optical system, such as a structure prism, is disposed immediately before the incident surface of the light guide 201, the similar effect can be acquired.

### Seventh Concrete Example of Coupling Section 103

Next, the seventh concrete example of the coupling section 103 is described while referring to FIG. 18. In the first to sixth concrete examples, the incident angle adjusting mechanism 133 is disposed and the incident angle of light rays to the light guide 201 is changed. However, as shown in FIG. 18, also, by changing an angle formed by the optical axis of the light guide 201 in a coupled state and the optical axis of the coupling section 103, it is possible to change the incident angle of light rays to the light guide 201.

Namely, as shown at an upper stage in FIG. 18, in the case where the coupling section 103 is coupled to the light guide 201 such that the optical axis of the coupling section 103 and the optical axis of the light guide 201 are coincident with each other, the incident angle of illumination light on the incident surface of the light guide 201 becomes a relatively small angle, and the irradiation region of the illumination light becomes narrow relatively. On the other hand, as shown at a lower stage in FIG. 18, in the case where the coupling section 103 is inclined obliquely relative to the light guide 201, the incident angle of illumination light on the incident surface of the light guide 201 becomes a relatively large angle, and the irradiation region of the illumination light becomes wide relatively.

Therefore, by controlling the inclined state of the coupling section 103 with the driving mechanism 107, it becomes possible to realize the function like the above.

In the above, while referring to FIG. 6 to FIG. 18, the constitution of the coupling section 103 included in the endoscope light source 10 according to the present embodiment has been described in detail.

<With Regard to Control Method of Endoscope Light Source>

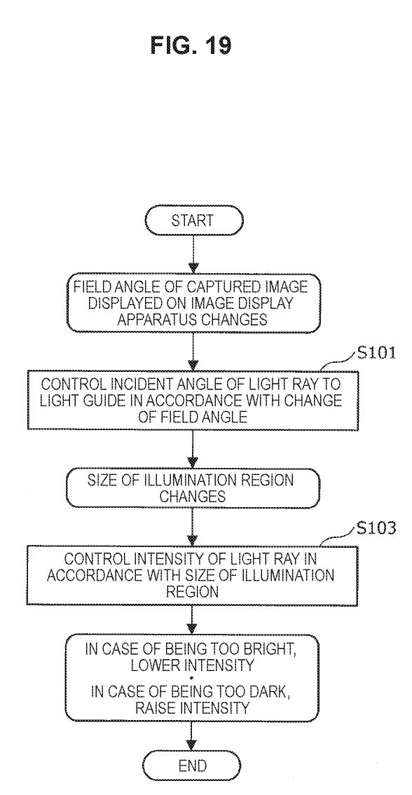

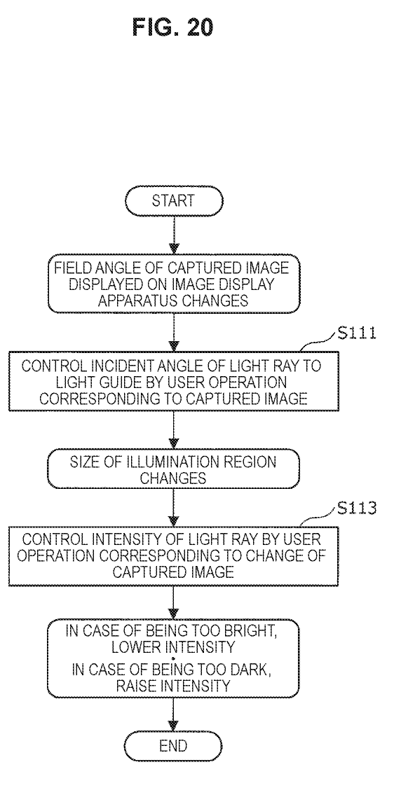

Successively, while referring to FIG. 19 and FIG. 20, a flow of a control method of the endoscope light source according to the present embodiment is described briefly. FIG. 19 is a flow diagram showing one example of a flow of a control method of the endoscope light source according to the present embodiment.

In advance of description of a control method of the endoscope light source, it is assumed that the field angle of a captured image displayed on the image display apparatus 205 has been changed owing to various kinds of operations performed by an operator of the endoscope apparatus 1 including the endoscope light source 10 according to the present embodiment.

In the image display apparatus 205, in the case where the field angle of the captured image being displayed has changed, the information indicating that the field angle of the captured image has changed is output to the control section 109 of the endoscope light source 10.

Upon acquisition of the information indicating that the field angle of the captured image has changed, from the image display apparatus 205, the control section 109 of the endoscope light source 10 refers to information with regard to the size of the changed field angle included in this information. Thereafter, the control section 109 controls the incident angle of light rays (illumination light) to the light guide 201 by making the incident angle adjusting mechanism 133 of the coupling section 103 etc. drive appropriately with the driving mechanism 107 (Step S101). With this, the size of the irradiation region of illumination light is changed in accordance with the field angle.

Subsequently, the control section 109 controls the intensity of light rays in accordance with the size of the irradiation region if needed (Step S103). That is, in the irradiation region after the changing, in the case where the irradiation region is too bright, the control section 109 controls the light source section 101 so as to lower the intensity of illumination light emitted from the light source section 101. Moreover, in the irradiation region after the changing, in the case where the irradiation region is too dark, the control section 109 controls the light source section 101 so as to increase the intensity of illumination light emitted from the light source section 101. With this, in accordance with the size of the irradiation region, the brightness of illumination light will be controlled appropriately.

Moreover, FIG. 20 is a flow diagram showing another example of the flow of the control method of the endoscope light source according to the present embodiment.

A captured image is displayed on the image display apparatus 205 by various kinds of operations performed by an operator of the endoscope apparatus 1 including the endoscope light source 10 according to the present embodiment performed. The operator of the endoscope apparatus 1 who checked the captured image controls the incident angle of light rays that enter the light guide 201 in the coupling section 103 via the control section 109 by executing various kind of user's operations (Step S111). With this, the size of the irradiation area of illumination light will change in accordance with the user's operations. Thereafter, the control section 109 also performs control of the intensity of light rays on the basis of the user's operations in accordance with a change of the captured image (Step S113). With this, the brightness of illumination light will be controlled appropriately.

In the above, while referring to FIG. 19 and FIG. 20, one example of the flow of the control method of the endoscope light source according to the present embodiment has been described briefly.

<With Regard to Modified Example of Endoscope Apparatus>

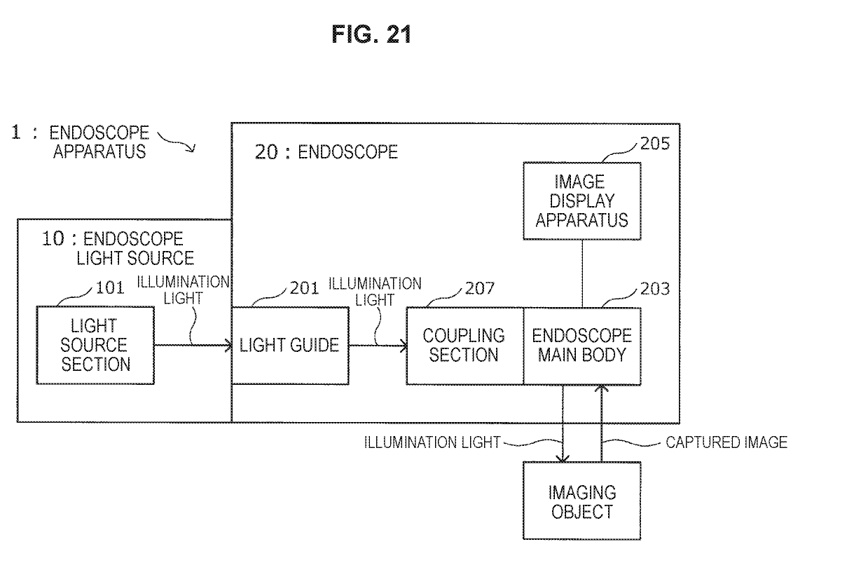

Next, while referring to FIG. 21, a modified example of the endoscope apparatus 1 according to the present embodiment is described briefly. FIG. 21 is an explanatory diagram showing schematically a modified example of the endoscope apparatus according to the present embodiment.

In the endoscope apparatus 1 according to the present embodiment shown in FIG. 1, the incident angle of illumination light that enters the endoscope 20 is controlled by the coupling section 103 disposed in the endoscope light source 10. However, the arrangement position of the coupling section 103 having the above function should not be limited to the example shown in FIG. 1, and, may be disposed in the inside of the endoscope 20.

That is, as shown schematically in FIG. 21, it is also possible to dispose a coupling section 207 having a constitution similar to the coupling section 103 at a position where illumination light is connected in the endoscope main body 203. At the position where illumination light is connected to the endoscope main body 203, in many cases, a bundle optical fiber similar to the light guide 201 is disposed. Then, the coupling section 207 having a constitution similar to the above-mentioned coupling section 103 may be connected to the bundle optical fiber (not shown) disposed in the endoscope main body 203. With this, it is possible to realize the similar effect as the case where the coupling section 103 is disposed in the endoscope light source 10.

In the above, while referring to FIG. 21, the modified example of the endoscope apparatus 1 according to the present embodiment has been described briefly.

(Conclusion)

As having described in the above, in the endoscope light source 10 according to the present embodiment, and the endoscope apparatus 1 using this endoscope light source 10, it becomes possible to make an image bright by making the luminance of the center portion of an illumination region higher than usual. With this, in the case of using a rigid endoscope, it is possible to realize a wide surgical space where sufficient brightness is secured, and the difficulty of surgery is lowered by reducing doctor's stress, whereby it is expected to improve the success rate of surgery.

Moreover, in the endoscope light source 10 according to the present embodiment and the endoscope apparatus 1 using this endoscope light source 10, since excessive illumination at a peripheral portion is eliminated, it is expected to reduce power consumption and to make the life of devices longer, and in addition, it is expected to reduce tissue damage by radiation heat.

Moreover, in the case where a flexible endoscope including such an endoscope light source 10 is used for observation of luminal organs, even if the luminal organ is located at a back side where the flexible endoscope cannot actually enter, it becomes possible to observe by a combination of an image magnifying view and a change of an illumination region, and it becomes possible to perform a still more exact medical examination.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.
(1)

An endoscope light source, including:

a light source section that emits light from at least one or more solid light sources;

a coupling section capable of connecting with a light guide connected to an endoscope; and

a control section that performs control so as to make an incident angle of a light ray that enters the light guide in the coupling section, changeable.
(2)

The endoscope light source according to (1), in which the solid light source is a light source having Etendue equal to or less than Etendue of the light guide.
(3)

The endoscope light source according to (1) or (2), in which a coupling optical system that couples the light ray with an incident angle having been controlled relative to the light guide, to the light guide is disposed in the coupling section.
(4)

The endoscope light source according to any one of (1) to (3), in which the light source section emits white light by mixing colors of light from two or more solid light sources.
(5)

The endoscope light source according to any one of (1) to (4), in which

a reflective optical system that reflects a light ray emitted from the light source section or a refractive optical system that refracts the light ray, and a coupling optical system that couples the light ray to the light guide are disposed in the coupling section, and

an incident angle of the light ray is changed by moving the reflective optical system or the refractive optical system so as to change a separation distance between an optical axis of the coupling optical system and an incident position of the light ray on an incident surface to the coupling optical system.
(6)

The endoscope light source according to any one of (1) to (5), in which an incident angle of the light ray is changed by changing an angle formed by an optical axis of the coupling section and an optical axis of the light guide.
(7)

The endoscope light source according to any one of (1) to (6), in which an incident angle of the light ray is changed by changing a beam size of the light ray on an incident surface of the light ray to the light guide.
(8)

The endoscope light source according to (7), in which

a coupling optical system that couples the light ray with an incident angle having been controlled relative to the light guide, to the light guide is disposed in the coupling section, and

a beam size of the light ray is changed by changing a magnification of the coupling optical system.
(9)

The endoscope light source according to (7), in which

a beam size converting mechanism that changes a beam size of light having entered the coupling section, is disposed in the coupling section, and

a beam size of the light ray is changed by driving the beam size converting mechanism.
(10)

The endoscope light source according to any one of (1) to (7), in which an incident angle of the light ray is changed by changing a divergent angle of the light ray emitted from the light source section.
(11)

The endoscope light source according to (10), in which

a diffusion plate is disposed in the coupling section or between the coupling section and the light source section, and

a divergent angle of the light ray is changed by changing the diffusion plate.
(12)

The endoscope light source according to (11), in which a divergent angle of the light ray is changed by performing at least any of replacement with the diffusion plate of a different kind and change of the number of the diffusion plates to be disposed.
(13)

The endoscope light source according to (10), in which

a multi lens array in which a plurality of lenses is arranged in an array form is disposed in the coupling section or between the coupling section and the light source section, and

a divergent angle of the light ray is changed by changing the multi lens array.
(14)

The endoscope light source according to (13), in which a divergent angle of the light ray is changed by performing at least any of replacement with the multi lens array of a different kind and change of the number of the multi lens arrays to be disposed.
(15)

The endoscope light source according to any one of (1) to (14), in which a light ray emitted from the light source section is propagated to the coupling section by a multi-mode optical fiber with a core diameter of 10 μm or more.
(16)

The endoscope light source according to any one of (1) to (15), in which in a case where a field angle when an image captured by the endoscope is displayed on a display screen, has changed, an incident angle of the light ray changes in accordance with a change of the field angle.
(17)

The endoscope light source according to (16), in which a size of an illumination region is changed in accordance with a change ratio of a size of the image on the display screen.
(18)

The endoscope light source according to (17), in which an intensity of a light ray emitted from the light source section is changed in accordance with a change of a size of the illumination region.
(19)

The endoscope light source according to any one of (1) to (18), in which the control section performs control so as to make an incident angle of a light ray that enters the light guide in the coupling section, changeable on a basis of an operation of a user.
(20)

A control method of an endoscope light source, including:

guiding a light ray emitted from a light source section that emits light from at least one or more solid light sources, to a coupling section capable of connecting with a light guide connected to an endoscope, and changing an incident angle of a light ray that enters the light guide in the coupling section.
(21)

An endoscope apparatus, including:

an endoscope that is inserted in an inside of an examination object, images an inside of the examination object, and propagates an obtained captured image to a display apparatus;

a light source section that emits light from at least one or more solid light sources as illumination light used when the endoscope images an inside of the examination object;

a coupling section capable of connecting with a light guide connected to the endoscope; and

a control section that performs control so as to make an incident angle of a light ray that enters the light guide in the coupling section, changeable.

### REFERENCE SIGNS LIST

1 endoscope apparatus
10 endoscope light source
20 endoscope
101 light source section
103, 207 coupling section
105 multi-mode optical fiber
107 driving mechanism
109 control section
111 memory section
121 solid light source
131 collimator lens
133 incident angle adjusting mechanism
135 coupling optical system
201 light guide
203 endoscope main body
205 image display apparatus

1. An endoscope light source, comprising:
a light source section that emits light from at least one or more solid light sources;
a coupling section capable of connecting with a light guide connected to an endoscope; and
a control section that performs control so as to make an incident angle of a light ray that enters the light guide in the coupling section, changeable.

2. The endoscope light source according to claim 1, wherein the solid light source is a light source having Etendue equal to or less than Etendue of the light guide.

3. The endoscope light source according to claim 1, wherein a coupling optical system that couples the light ray with an incident angle having been controlled relative to the light guide, to the light guide is disposed in the coupling section.

4. The endoscope light source according to claim 1, wherein the light source section emits white light by mixing colors of light from two or more solid light sources.

5. The endoscope light source according to claim 1, wherein
a reflective optical system that reflects a light ray emitted from the light source section or a refractive optical system that refracts the light ray, and a coupling optical system that couples the light ray to the light guide are disposed in the coupling section, and
an incident angle of the light ray is changed by moving the reflective optical system or the refractive optical system so as to change a separation distance between an optical axis of the coupling optical system and an incident position of the light ray on an incident surface to the coupling optical system.

6. The endoscope light source according to claim 1, wherein an incident angle of the light ray is changed by changing an angle formed by an optical axis of the coupling section and an optical axis of the light guide.

7. The endoscope light source according to claim 1, wherein an incident angle of the light ray is changed by changing a beam size of the light ray on an incident surface of the light ray to the light guide.

8. The endoscope light source according to claim 7, wherein
a coupling optical system that couples the light ray with an incident angle having been controlled relative to the light guide, to the light guide is disposed in the coupling section, and
a beam size of the light ray is changed by changing a magnification of the coupling optical system.

9. The endoscope light source according to claim 7, wherein
a beam size converting mechanism that changes a beam size of light having entered the coupling section, is disposed in the coupling section, and
a beam size of the light ray is changed by driving the beam size converting mechanism.

10. The endoscope light source according to claim 1, wherein an incident angle of the light ray is changed by changing a divergent angle of the light ray emitted from the light source section.

11. The endoscope light source according to claim 10, wherein
a diffusion plate is disposed in the coupling section or between the coupling section and the light source section, and
a divergent angle of the light ray is changed by changing the diffusion plate.

12. The endoscope light source according to claim 11, wherein a divergent angle of the light ray is changed by performing at least any of replacement with the diffusion plate of a different kind and change of the number of the diffusion plates to be disposed.

13. The endoscope light source according to claim 10, wherein
a multi lens array in which a plurality of lenses is arranged in an array form is disposed in the coupling section or between the coupling section and the light source section, and
a divergent angle of the light ray is changed by changing the multi lens array.

14. The endoscope light source according to claim 13, wherein a divergent angle of the light ray is changed by performing at least any of replacement with the multi lens array of a different kind and change of the number of the multi lens arrays to be disposed.

15. The endoscope light source according to claim 1, wherein a light ray emitted from the light source section is propagated to the coupling section by a multi-mode optical fiber with a core diameter of 10 μm or more.

16. The endoscope light source according to claim 1, wherein in a case where a field angle when an image captured by the endoscope is displayed on a display screen, has changed, an incident angle of the light ray changes in accordance with a change of the field angle.

17. The endoscope light source according to claim 16, wherein a size of an illumination region is changed in accordance with a change ratio of a size of the image on the display screen.

18. The endoscope light source according to claim 17, wherein an intensity of a light ray emitted from the light source section is changed in accordance with a change of a size of the illumination region.

19. The endoscope light source according to claim 1, wherein the control section performs control so as to make an incident angle of a light ray that enters the light guide in the coupling section, changeable on a basis of an operation of a user.

20. A control method of an endoscope light source, comprising:
guiding a light ray emitted from a light source section that emits light from at least one or more solid light sources, to a coupling section capable of connecting with a light guide connected to an endoscope, and changing an incident angle of a light ray that enters the light guide in the coupling section.

21. An endoscope apparatus, comprising:
an endoscope that is inserted in an inside of an examination object, images an inside of the examination object, and propagates an obtained captured image to a display apparatus;
a light source section that emits light from at least one or more solid light sources as illumination light used when the endoscope images an inside of the examination object;
a coupling section capable of connecting with a light guide connected to the endoscope; and
a control section that performs control so as to make an incident angle of a light ray that enters the light guide in the coupling section, changeable.
